(19) United States
(12) Patent Application Publication
(10) Pub. No.: US 2013/0089640 A1
(43) Pub. Date: Apr. 11, 2013

(54) SOLID ENZYME FORMULATIONS AND METHOD FOR PRODUCTION THEREOF

(71) Applicant: BASF SE, Ludwigshafen (DE)

(72) Inventors: Markus Lohscheidt, Heidelberg (DE); Roland Betz, Niederkirchen (DE); Jörg Braun, Essingen (DE); Wolf Pelletier, Ottersheim (DE)

(73) Assignee: BASF SE, Ludwigshafen (DE)

(21) Appl. No.: 13/683,644

(22) Filed: Nov. 21, 2012

Related U.S. Application Data
(63) Continuation of application No. 12/282,254, filed on Jan. 20, 2009.

(30) Foreign Application Priority Data
Mar. 10, 2006 (EP) 06004998.8

(51) Int. Cl. C12N 9/98; A23K 1/165

(52) U.S. Cl. CPC C12N 9/98; A23K 1/1653
USPC 426/63; 426/61

(57) ABSTRACT

The present invention relates to novel solid enzyme formulations comprising mixtures of at least one salt-stabilized enzyme composition, at least one particulate support and at least one hydrophobic liquid. In addition, the invention relates to methods for producing such solid enzyme formulations and also animal feed, foods and food supplements which comprise such enzyme formulations.

The present invention relates to novel solid enzyme formulations comprising mixtures of at least one salt-stabilized enzyme composition, at least one particulate support and at least one hydrophobic liquid. In addition, the invention relates to methods for producing such solid enzyme formulations and also animal feed, foods and food supplements which comprise such enzyme formulations.

### BACKGROUND OF THE INVENTION

From the prior art, numerous solid enzyme compositions are known which are produced, for example, by spray-drying liquid enzyme solutions. It is further known that the enzyme stability in such spray-drying processes can be significantly increased by adding stabilizing salts, such as, for example, magnesium sulfate. This therefore produces in this manner solid enzyme compositions which also, even after spray drying, have a high enzyme activity percentage. For example, in EP-A-0 758 018, storage-stable and processing-stable solid enzyme compositions are described which are obtained by drying a solution comprising at least one enzyme and a water-soluble inorganic salt. The enzyme compositions described there are preferably used as additive for solid animal feed compositions.

For the production of such enzyme-additized animal feed compositions, it is desirable that the enzyme is distributed as uniformly as possible in the finished feed preparation. Since the dry enzyme preparations comprise the enzyme in high concentration, addition of significantly less than 1% by weight, based on the total weight of the feed composition, is generally completely sufficient to provide the desired enzyme activity for the feed composition. The lower the required amount of enzyme to be added, the more difficult it is, however, to achieve uniform distribution of the enzyme activity in the finished feed preparation. The same difficulty is of course also observed in the production of foods and food supplements to which highly concentrated solid enzyme compositions are to be added distributed as uniformly as possible.

The object of the invention is therefore to find a way which makes it possible to bring highly concentrated solid enzyme compositions which essentially comprise only enzyme and stabilizing support into a form which ensures uniform and reproducible dosage to foods and feeds. At the same time the invention should also ensure that the formulations used therefor have good processing properties, such as reduced dusting tendency, good rheological behavior and narrow particle size distribution.

### SUMMARY OF THE INVENTION

Said object has surprisingly been achieved by providing a solid enzyme formulation which is obtained by mixing a particulate, salt-stabilized enzyme composition, a particulate support, and also a hydrophobic liquid. In particular, it was surprising that the inventively produced solid formulations are particularly easy to handle, since they exhibit high separation stability, extremely low dusting tendency and, despite addition of hydrophobic liquid, an excellent rheological behavior.

### DESCRIPTION OF FIGURES

FIG. 1 shows on the basis of a flow diagram a preferred embodiment of the present invention, in particular the production of a solid xylanase formulation. For this, a xylanase-comprising liquid concentrate is mixed with magnesium sulfate, dried in a spraying apparatus to give a xylanase-comprising stabilized powder and simultaneously agglomerated, particles, for example having a size in the range from 50 to 250 μm, being able to be obtained. In the next step, the xylanase-comprising dry powder is mixed with a solid organic support and simultaneously or subsequently sprayed with soybean oil. This produces in this manner a xylanase-comprising formulation having low dusting tendency and high separation stability.

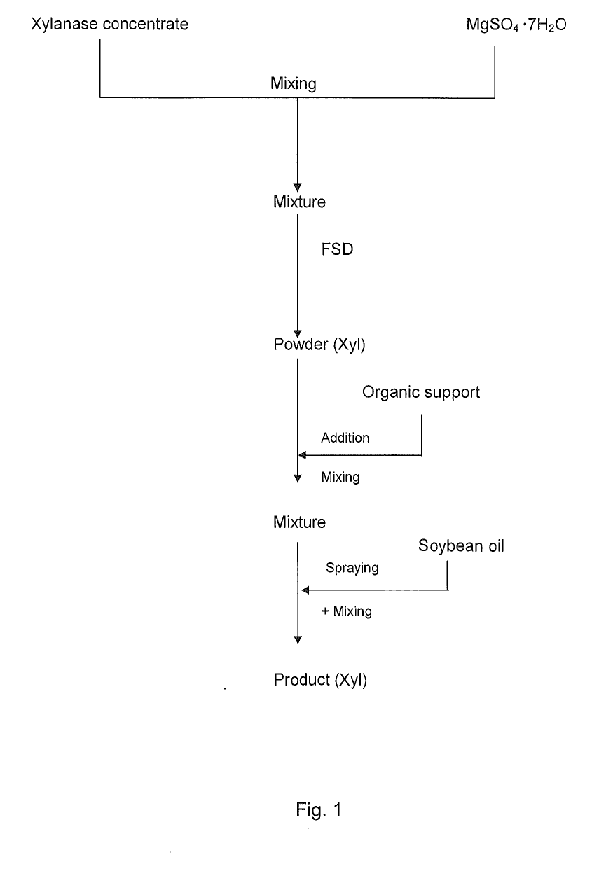

FIG. 2 illustrates the production of further inventively preferred solid enzyme formulations which comprise, in different embodiments, a mixture of xylanase and glucanase. According to method variant (a), the method proceeds from a liquid mixed concentrate of glucanase and xylanase, whereas in method variant (b), the method first proceeds from a liquid glucanase concentrate. According to method variant (a), the mixed concentrate of glucanase and xylanase as described above for FIG. 1 is dried and mixed with an organic support and sprayed with soybean oil. According to method variant (b), in contrast, first a liquid glucanase concentrate is processed to give a glucanase-comprising powder in a similar manner to that described above for the xylanase concentrate. This powder is mixed with a xylanase powder prepared according to FIG. 1. At the same time it is mixed with the organic support and sprayed with soybean oil, likewise a xylanase- and glucanase-comprising enzyme formulation being produced. The resultant glucanase- and xylanase-comprising solid enzyme formulations produced are also distinguished by very low dusting tendency and high separation stability.

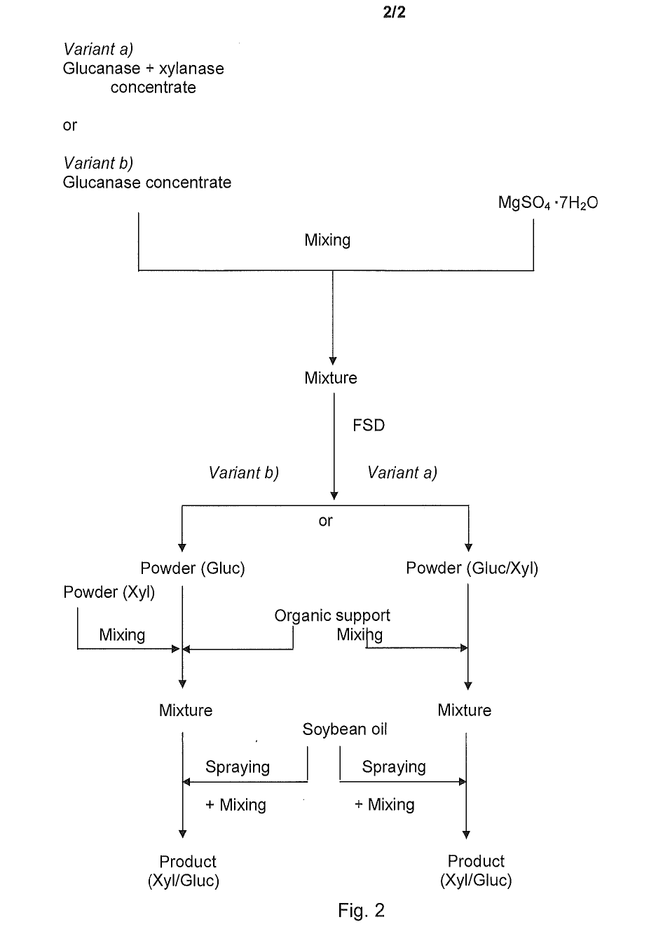

### DETAILED DESCRIPTION OF THE INVENTION

### a) Preferred Embodiments of the Invention

The invention relates to solid enzyme formulations comprising a mixture of a) at least one particulate, granular enzyme composition of at least one enzyme and at least one organic or inorganic salt of a monovalent or divalent metal cation with b) at least one particulate inorganic or organic physiologically compatible support and c) at least one hydrophobic liquid having adhesive properties, and in particular a hydrophobic liquid having a melting point in the range from −60° C. to 30° C., in particular from −50 to 0° C., such as, for example, from −40 to −5° C., or from −30 to −10° C.

The invention relates in particular to enzyme formulations comprising

a) a particulate enzyme composition comprising an enzyme in a mixture with at least one organic or inorganic salt of a monovalent or divalent cation; or
b) a particulate enzyme composition comprising at least two enzymes which are different from one another in a mixture with at least one organic or inorganic salt of a monovalent or divalent cation; or
c) at least two particulate enzyme compositions which are different from one another, the two compositions differing in that they comprise at least one different enzyme, the enzymes in each composition being present in a mixture with at least one organic or inorganic salt of a monovalent or divalent cation.

In particular, the invention relates to enzyme formulations, the ratio of the median particle diameters of support to enzyme composition being in the range of from about 0.125 to 8, in particular 0.25 to 4, or 0.5 to 2 or 1 to 1.5. The median particle size of enzyme composition used and support used should be in the range of from about 50 to 500 μm, or 150 to 350 μm. Expediently, the mixing ratio of enzyme composition and support is set in the range from about 1:1000 to 1:5 parts by weight, or 1:500 to 1:10, or 1:100 to 1:20.

The fraction of hydrophobic liquid is 0.1 to 5, 0.2 to 2, 0.3 to 1.5, or 0.3 to 0.7% by weight, based on the total weight of the enzyme formulation.

In the enzyme compositions used according to the invention, the salt fraction is in the range from 1 to 30% by weight, 5 to 25% by weight, or 10 to 20% by weight, based on the total weight of the enzyme composition.

The percentage fraction of enzyme protein in the enzyme composition is about 0.01 to 99% by weight, such as, for example, 0.01 to 80% by weight, 10 to 80% by weight, 20 to 75% by weight, or 30 to 60% by weight.

In addition to at least one enzyme and at least one salt, the enzyme composition can, moreover, comprise further components. These can serve as binder (e.g. polymers or sugars), as filler (e.g. lime, loam, carbohydrates, sugars, starch), as dye or further stabilizer. Such further components are known per se from the prior art and are familiar to those skilled in the art.

The residual moisture of the enzyme mixture is according to the invention in a range from 5 to 30% by weight, such as, for example, from 5 to 20% by weight, or from 7 to 16% by weight.

The invention is not limited to any defined enzymes. In particular, however, the usable enzymes are selected from hydrolases (EC 3.), in particular glycosidases (EC 3.2.1), peptidases (EC 3.4) and especially xylanases, glucanases (hemicellulases), cellulases, proteases, keratinases, amylases, peptidases and mixtures thereof.

In preferred enzyme formulations, the enzyme is selected from endo-1,4-β-xylanases (EC 3.2.1.8), endo-1,4-β-glucanases (EC 3.2.1.4) and mixtures thereof.

The invention also relates to enzyme formulations which have at least one further of the following properties:
a) gravimetric dusting value (determined according to a method described in the examples) in the range from 0 to 0.5, or 0.001 to 0.3, or 0.01 to 0.2% by weight;
b) bulk density in the range from 200 to 700, 300 to 500, or 350 to 450 g/l (defined as specified in DIN EN ISO 60)
c) flowability (determined by Schulze ring shear test) having an ff_{c} value in the range from 3 to 30, 5 to 15, or 6 to 10.

The invention relates in particular to enzyme formulations, the formulation comprising a mixture of
a) at least one enzyme composition, the enzyme component of which is selected from xylanases, glucanases and mixtures thereof according to aforesaid definition in a mixture with magnesium sulfate, the magnesium sulfate fraction being about 5 to 25% by weight, or 15 to 20% by weight, based on the total weight of the dry enzyme composition;
b) at least one wheat semolina bran support, the mixing ratio of enzyme composition to support being in the range from 1:5 to 1:500, or 1:10 to 1:100;
c) vegetable oil in a fraction of about 0.1 to 1% by weight, or 0.3 to 0.6% by weight based on the final weight of the enzyme formulation,
   the median particle size of enzyme composition and support being in the range from about 100 to 500, or 150 to 350 μm, and the xylanase fraction being about 3000-30 000, or 5200 to 18 000, or 5400 to 9000 TXU/g of formulation and the glucanase fraction being about 2000 to 20 000, or 2200 to 10 000 TGU/g of formulation. The percentage fraction of the xylanase is about 1-20% by weight, preferably 2-10% by weight, and in particular 2.5-5% by weight, and of glucanase about 0.01-10% by weight, preferably 0.1-6% by weight, and in particular 0.2-2% by weight.

Particular preference is given to formulations comprising an enzyme composition of the above-described type, the enzyme component of which is a xylanase.

Particular preference is given to formulations comprising an enzyme composition of the above-described type, the enzyme component of which is a glucanase.

Particular preference is given to formulations comprising an enzyme composition of the above-described type, the enzyme component of which is a mixture of xylanase and glucanase.

Particular preference is given to formulations comprising two enzyme compositions of different enzymes, the one enzyme component being a glucanase and the other a xylanase.

The invention also relates to methods for producing solid enzyme formulations according to aforesaid definition, at least one particulate enzyme composition comprising at least one enzyme and at least one organic or inorganic salt of a monovalent or divalent metal cation being mixed with a particulate inorganic or organic physiologically compatible support and the mixture being wetted with a hydrophobic liquid (having a melting point in the range from −60 to 30° C. according to aforesaid definition).

In particular the invention relates to methods in which
a) a particulate enzyme composition comprising an enzyme, in particular xylanase or glucanase, is in a mixture with at least one organic or inorganic salt of a monovalent or divalent cation is provided; or
b) a particulate enzyme composition comprising at least two enzymes which are different from one another, selected from xylanase and glucanase, provided in a mixture with at least one organic or inorganic salt of a monovalent or divalent cation; or
c) at least two particulate enzyme compositions which are different from one another are provided, the two compositions differing in that they comprise at least one different enzyme, the enzymes in each composition being present in a mixture with at least one organic or inorganic salt of a monovalent or divalent cation.

Preferred methods are those in which the enzyme composition is obtained by spray drying or by spray drying and agglomeration of an enzyme-comprising liquid in which at least one organic or inorganic salt of a monovalent or divalent cation is taken up.

In addition, preferred methods are those in which at least two enzyme compositions of enzymes which are different from one another are obtained by spray drying or by spray drying and agglomeration of at least two different enzyme-comprising liquids in which at least one organic or inorganic salt of a monovalent or divalent cation is taken up, and
a) each of the at least two enzyme compositions is mixed with a particulate inorganic or organic support, or
b) a particulate inorganic or organic support is mixed with the at least two enzyme compositions; and
   the mixture produced according to variant a) or variant b) is wetted with a hydrophobic liquid.

The enzyme-comprising liquid used comprises at least one xylanase, at least one glucanase or a mixture thereof.

In particular, the salt fraction in the enzyme composition used is in the range from 1 to 30% by weight, or at about 10 to 25% by weight or 15 to 20% by weight, based on the total weight of the enzyme composition.

In addition, use is made, in particular, of a support and an enzyme composition the ratio of median particle diameters of which is in the range from about 0.125 to 8, preferably 0.25 to 4, or 0.5 to 2, or 1 to 1.5.

The median particle size of enzyme composition used and support used is in the range from about 50 to 500 μm, or 150 to 350 μm. The mixing ratio of enzyme composition and support is in the range from about 1:1000 to 1:5, or 1:500 to 1:10, or 1:100 to 1:20.

The median particle size can, according to the size of the particles, be determined either by means of sieving analysis (e.g. using a shaking sieve machine type Vibro VS 1000 from Retsch), or else by laser diffraction (e.g. using a Mastersizer from Malvern).

The fraction of hydrophobic liquid is 0.1 to 5% by weight, or 0.2 to 2, 0.3 to 1.5, or 0.3 to 0.7% by weight, based on the total weight of the enzyme formulation.

The invention relates in particular to a method for producing a solid enzyme formulation comprising at least one enzyme selected from xylanases, glucanases and mixtures thereof,
a) at least one enzyme-comprising liquid being spray dried or spray dried and agglomerated to give at least one enzyme composition, the enzyme component of which being selected from xylanases, glucanases and mixtures thereof, and this enzyme component being present in the liquid in a mixture with magnesium sulfate, and the magnesium sulfate fraction being about 10 to 25% by weight, based on the total weight of the dry enzyme composition;
b) the resultant enzyme composition being mixed with a particulate inorganic or organic support; and
c) the enzyme/support mixture being wetted with a hydrophobic liquid having a melting point between −60 and 30° C.

Preferred method variants comprise
a) a particulate enzyme composition comprising at least one xylanase in a mixture with magnesium sulfate being provided; or
b) a particulate enzyme composition comprising at least one glucanase in a mixture with magnesium sulfate being provided; or
c) a particulate enzyme composition comprising at least one xylanase and at least one glucanase in a mixture with magnesium sulfate being provided; or
d) at least two particulate enzyme compositions which are different from one another being provided, one of the compositions comprising at least one xylanase and the other of the compositions comprising at least one glucanase, the enzymes in each composition being present in a mixture with magnesium sulfate.

In a particular embodiment of the method, an enzyme composition is mixed with at least one wheat semolina bran support, the mixing ratio of enzyme composition to support being in the range from 1:5 to 1:500, or 1:10 to 1:100.

During mixing, in particular vegetable oil is added in a fraction of from about 0.1 to 1% by weight, or 0.3 to 0.6% by weight, based on the final weight of the enzyme formulation. The median particle size of enzyme composition used and support used is in particular in the range from about 100 to 500 μm, or 150 to 40 μm, and the xylanase fraction is about 5000-30 000, or 5200 to 10 000, or 5400 to 9000 TXU/g of formulation and/or the glucanase fraction is about 2000 to 10 000, or 2200 to 6000 TGU/g of formulation.

The invention also relates to the use of a dry enzyme formulation according to aforesaid definition for producing a food, food supplement or an animal feed.

The invention also relates to animal feeds, foods or food supplements comprising a dry enzyme formulation according to aforesaid definition; in particular animal feeds comprising the inventive enzyme formulation in a fraction of from about 0.001 to 1% by weight.

### b) Enzymes

The enzymes used according to the invention are not subject to any limitations and can be either of natural or recombinant origin. The enzymes can be enzymes from plants, from fungi, from bacteria or yeasts. Preference is given to enzymes from microbiological sources such as bacteria, yeasts or fungi. The enzyme can be obtained from the respective microorganism by known techniques which typically comprise fermentation of the enzyme-producing microorganism in a suitable nutrient medium and subsequent isolation of the enzyme or enzyme concentrate from the fermentation medium by standard techniques.

If required, to set the pH of the enzyme solution or of the enzyme concentrate, conventional substances such as buffers, bases, acids, can be added to the formulations; preferred pHs are 3.5 to 7, particularly preferably 3.5 to 5, and in particular 4 to 4.5.

In addition, use can be made of enzyme mutants or enzymes which exhibit an elevated heat stability, such as, for example, proposed in the WO's 95/2997, 97/00020, 97/20920, 97/22691, 98/28410 or 03/062409.

Preferably, however, use is made according to the invention as enzymes of polypeptides having xylanase activity, polypeptides having glucanase activity and mixtures thereof.

b1) Polypeptides Having Xylanase Activity

These are enzymes of class EC 3.2. 1.8 having the official name endo-1,4-beta-xylanase. The systematic name is 1,4-beta-D-xylanxylanohydrolase. Other names likewise in use are: endo-(1-4)-beta-xylanase; (1-4)-beta-xylan 4-xylanohydrolase; endo-1,4-xylanase; xylanase; beta-1,4-xylanase; endo-1,4-xylanase; endo beta-1,4-xylanase; endo-1,4-beta-D-xylanase; 1,4-beta-xylan xylanohydrolase; beta-xylanase; beta-1,4-xylan xylanohydrolase; endo-1,4-beta-xylanase; beta-D-xylanase. The enzyme catalyzes the endohydrolysis of 1,4-beta-D-xylosidic bonds in xylans.

The xylanase can be derived, for example, from bacteria, such as, for example, those of the genera Clostridium, Streptomyces, Paenibacillus, Pseudomonas, Thermoascus, Thermotoga, Bacillus, and, for example, xylanases from the following strains Bacillus halodurans, Bacillus pumilus, Bacillus agaradhaerens, Bacillus circulans, Bacillus polymyxa, Bacillus sp., Bacillus stearothermophilus, or Bacillus subtilis.

Fungal xylanases are derived, for example, from yeasts and filamentous fungi, such as, for example, from the following genera: Aspergillus, Aureobasidium, Emericella, Fusarium, Gaeumannomyces, Humicola, Lentinula, Magnaporthe, Neocallimastix, Nocardiopsis, Orpinomyces, Paecilomyces, Penicillium, Pichia, Saccharomyces, Schizophyllum, Talaromyces, Thermomyces, Trichoderma, such as, for example, Talaromyces emersonii.

The xylanase activity is determined in a manner known per se and is described, for example, in Engelen et al., Journal of AOAC International Vol. 79, No. 5, 1019 (1996). In contrast to the method described there, instead of the xylan substrate from oat spelts (Serva Feinbiochemia GmbH u. Co., Heidelberg), use is made of arabinoxylan from wheat (Megazyme, article P-WAXY, Ireland). The substrate solution is prepared fresh in each case by dissolving 1000 g of arabinoxylan lump-free in 100.00 ml of water over a period of at least 12 hours.

b2) Polypeptides Having Glucanase Activity

Endoglucanases are classified as EC 3.2.1.4 and are frequently called cellulases. Other names are endo-glucanase, endo-1,4-beta-glucanase, cellulase A or carboxymethylcellulase. The enzymes catalyze the endohydrolysis of 1,4-beta-D-glucosidic bonds in cellulose and also the 1,4-links in beta-D-glucans which in addition comprise 1,3-links.

The glucanase can be derived, for example, from bacteria, such as, for example, from those of the genera Bacillus, Clostridium, Paenibacillus, Pseudomonas, Streptomyces, Thermoascus, Thermotoga. Fungal glucanases are derived, for example, from yeasts and filamentous fungi, such as, for example, from the following genera: Aspergillus, Aureobasidium, Emericella, Fusarium, Gaeumannomyces, Humicola, Lentinula, Magnaporthe, Neocallimastix, Nocardiopsis, Orpinomyces, Paecilomyces, Penicillium, Pichia, Saccharomyces, Schizophyllum, Talaromyces, Thermomyces, Trichoderma, such as, for example, Talaromyces emersonii.

The glucanase activity is determined in a manner known per se and is described, for example, in Engelen et al., Journal of AOAC International Vol. 79, No. 5, 1019 (1996). In contrast to the method described there, instead of the beta-glucan substrate from barley (Sigma Chemical Co., St. Louis, Mo.: No. G-6513), use is made of beta-glucan from barley (Megazyme, article P-BGBM, Ireland). The substrate solution is prepared freshly in each case firstly by suspension of 0.750 g of glucan in 20 ml of water and subsequently dissolution by adding 20 ml of sodium hydroxide solution (2 mol/l) with stirring for 15 minutes. 42.5 ml of citric acid solution (1 mol/l) are added, the pH is adjusted to 3.50+/−0.03 at 40.0° C.+/−0.1° C. using sodium hydroxide solution (2 mol/l) or citric acid solution (1 mol/l). After cooling to room temperature, the mixture is made up to 100.00 ml with water.

### c) Stabilizing Salts

Examples of suitable stabilizing additives which may be mentioned are inorganic or organic salts.

In particular these are metal salts, in particular alkali metal and alkaline earth metal salts of organic acids, such as, for example, Mg, Ca, Zn, Na, K salts of monovalent or divalent carboxylic acids having 1 to 8 carbon atoms, such as, for example, citrates, acetates, formates and hydrogenformates, in addition inorganic salts, such as, for example, Mg, Ca, Zn, Na, K sulfates, carbonates, silicates or phosphates; alkaline earth metal oxides, such as CaO and MgO; inorganic buffering agents, such as alkali metal hydrogenphosphates, in particular sodium and potassium hydrogenphosphates, such as, for example, K₂HPO₄, KH₂PO₄ and Na₂HPO₄. Particularly preferably, use is made of the following salts in the weight fractions given based on the enzyme composition:
zinc sulfate (0.5 to 10, or 3 to 8% by weight)
calcium sulfate (1 to 30, or 10 to 25% by weight)
magnesium sulfate (5 to 30, or 10 to 25% by weight)
sodium sulfate (1 to 30, or 10 to 20% by weight)

### d) Suitable Supports

Examples of support materials are carbohydrates, in particular sugars and also starches, for example from corn, rice, potatoes, wheat and cassava; modified starches, for example octenyl succinate anhydride; cellulose and microcrystalline cellulose; inorganic minerals or loam, for example clay, coal, kieselguhr, silicic acid, talc and kaolin; semolina, for example wheat semolina, brans, for example wheat bran or wheat semolina, flours; salts such as metal salts, in particular alkali metal and alkaline earth metal salts of organic acids, for example Mg, Ca, Zn, Na, K citrate, acetate, formate and hydrogenformates, inorganic salts, for example Mg, Ca, Zn, Na, K sulfates, carbonates, silicates or phosphates; alkaline earth metal oxides such as CaO and MgO; inorganic buffering agents such as alkali metal hydrogenphosphates, in particular sodium and potassium hydrogenphosphates, for example K₂HPO₄, KH₂PO₄ and Na₂HPO₄.

### e) Suitable Hydrophobic Liquids

Examples of suitable hydrophobic liquids which may be mentioned are:

In principle all hydrophobic liquids (having a melting point in the range from −60 to 30° C. which have a hydrophobic molecule moiety) are usable provided that they are suitable as food or feed additive. Preference is given to naturally occurring plant or animal liquids such as phospholipids and mono-, di- and triacylglycerides and mixtures thereof.

Nonlimiting examples which may be mentioned are soybean lecithin, vegetable oils, such as, for example, sunflower oil, corn germ oil, soybean oil, palm oil, rapeseed oil, palm kernel oil, cottonseed oil, peanut oil, babassu oil, thistle oil and also animal oils, such as, for example, fish oil.

### f) Production of the Formulation

The inventive enzyme formulations are produced making use of methods known per se of the prior art, such as, for example, described in Mollet et al., Formulierungstechnik [Formulation technique], 2000, Verlag Wiley-VCH, Weinheim, or Heinze, Handbuch der Agglomerationstechnik [Handbook of agglomeration technique], 2000, Verlag Wiley-VCH, Weinheim.

f1) Drying

For producing the salt-stabilized, preferably agglomerated, enzyme compositions by drying, various technologies come into consideration, such as, in particular
spray drying
fluidized-bed granulation
fluidized-bed agglomeration
fluidized spray dryer (FSD) technology
Procell technology from Glatt (WO 2004/108911)

Drying can be performed continuously or batchwise. If appropriate, the dried product, after drying, must still be sieved, ground or agglomerated. Combinations of said steps are also possible.

The enzyme solution used according to the invention for spray drying or agglomeration comprises at least one enzyme usable as food additive or feed additive, dissolved or suspended in an aqueous phase such as, for example, the enzyme concentrate which can be obtained from the production process comprising fermentation and workup. The solution has a protein fraction in the range from about 1 to 50% by weight, preferably about 10 to 35% by weight, based on the total weight of the solution. The pH is generally in the range from about 3 to 9. In addition to the abovementioned salt-form enzyme stabilizers such as, for example, alkali metal or alkaline earth metal salts, such as sodium sulfate or magnesium sulfate, the solution can if appropriate comprise other conventional additives. Examples which may be mentioned are: buffers, such as, for example, phosphate buffers; solubilizers, such as, for example, ethanol or surface-active agents and the like.

In the event that the adhesive properties of the enzyme solution do not suffice to ensure stable sticking-together of the particles after spraying, the use in addition of a binder is advantageous. This avoids the agglomerates from disintegrating again on drying. In such cases it is preferred to spray into the fluidized bed a binder which is soluble or dispersible in aqueous medium. The binder can be sprayed in either dissolved in the enzyme solution to be sprayed in, or separately therefrom, simultaneously or offset in time. Examples of suitable binders which may be mentioned are: solutions of carbohydrates, such as, for example, glucose, sucrose, dextrins, inter alia, sugar alcohols, such as, for example, mannitol, or polymer solutions such as, for example, solutions of hydroxypropylmethylcellulose (HPMC), polyvinylpyrrolidone (PVP), polyvinyl alcohol (PVA), ethoxylated cellulose (EC), ethylcellulose or propylcellulose. Via targeted selection of amount and adhesive properties of the binder sprayed in, agglomerates of different size and strength can be produced.

If the binder is sprayed on in a mixture with the enzyme, the binder fraction is usually in the range from about 0.5 to 20% by weight, preferably about 1 to 10% by weight, based on the total weight of the solution.

If the binder is sprayed on as a separate solution, the binder fraction of the solution is in the range from about 1 to 30% by weight, based on the total weight of the solution. The binder in this case is likewise dissolved in an aqueous medium, preferably sterile demineralized water. Conventional additives such as, for example, buffers or solubilizers can likewise be present.

According to the invention, the fraction of the binder in the end product (that is the enzyme composition) is 0 to about 20% by weight, for example about 1 to 6% by weight. The optimum amount is also dependent on the type of binder selected.

The spray drying of liquid enzyme preparations can be carried out in a conventional manner. For this the enzyme solution is pumped to the atomizer in the spraying tower. The atomization is performed, for example, by means of a pressure nozzle (single-fluid nozzle), a twin-fluid nozzle or a centrifugal atomizer. The droplets are dried by a hot air stream passed into the spray dryer. When centrifugal atomizers are used, the drying is preferably performed in co-current flow. With nozzles, the drying can also be performed in counter-current flow or mixed-current flow. The powder can be discharged at the tower or it is entrained by the air stream and separated off in a cyclone and/or filter. Depending on product and procedure, post-drying may be required which can proceed in an internal fluidized bed flanged onto the spray dryer, or an external fluidized bed.

The spray-dried product can be subsequently agglomerated in a fluidized bed. For this purpose pulverulent material, for example enzyme powder obtained by aforesaid spray drying, is charged into a fluidized-bed dryer. The swirling is performed, for example, by feeding preheated air. An enzyme-comprising solution, for example, or a binder solution is sprayed onto the fluidized bed, as a result of which the charged powder is wetted with this solution and increasingly agglomerated owing to its adhesive properties. Spraying into the fluidized bed can proceed from the top (top spray method) or the bottom (bottom spray method). At the same time, simultaneously or semicontinuously, that is timed at intervals, a subquantity of agglomerate is discharged from the fluidized bed. The discharge is classified, using, for example, a sieve. Coarse material produced can be ground and continuously recirculated to the fluidized bed. Fine fractions, such as, for example, from the exhaust air filter system, can likewise be continuously recirculated.

According to a further method variant, the production of the inventive enzyme agglomerate can proceed continuously, more precisely with continuous feed of a dry pulverulent charge, such as, for example, a dry enzyme powder, into the fluidized-bed dryer. Particularly suitable dryers for this are fluidized-bed dryers having a plurality of spray zones and of appropriate drying zones. In the first zone, dry enzyme powder is charged, swirled, and enzyme solution and/or binder sprayed in. The agglomerate formed in this zone is transferred to the next zone. Into this, and if appropriate into one or more further zones, likewise enzyme solution ad/or binder solution of identical or different composition can be sprayed in. The water of the enzyme solution or binder solution sprayed on is removed by a feed air stream which is common for all zones, or separate feed air streams which are appropriately heated. In one or more of the last zones, post-drying can further be carried out. Here is also situated the product discharge. The workup of the product is performed as described above.

A further preferred method variant comprises a spray drying of enzyme solution, coupled with subsequent agglomeration of the spray-dried enzyme powder. This can be carried out batchwise or continuously. The continuous procedure is preferred.

Such methods can be carried out using conventional spray-drying plants. However, they can advantageously be carried out in apparatuses which are known as FSD (Fluidized Spray Dryer), SBD (Spray Bed Dryer) or MSD (Multi Stage Dryer).

The resultant fine fraction of the powder can in this case be reincorporated into the process as early as in the spray dryer, if it is recirculated, for example after precipitation in a cyclone or filter, back to the moist zone of the dryer. The actual agglomeration then takes place in a further stage in a fluidized bed. This stage can be integrated into the spray dryer (internal fluidized bed) or it can be carried out in a separate apparatus (additional fluidized bed). Into the fluidized bed there can be injected, if required, with simultaneous drying, further enzyme solution, an enzyme solution which in addition comprises binder, or only binder in dissolved or dispersed form, in order to support the agglomeration. Examples of suitable binders for the agglomeration are hydroxypropylmethylcellulose, polyvinylpyrrolidone, polyethylene glycols and block polymers of polyoxyethylene and polyoxypropylene. Preferably, the process parameters are set, however, in such a manner that no further addition for agglomerate production is required. The composition and amount of the liquids injected depend on the adhesive properties of the sprayed solution, the agglomerate size to be achieved and the process conditions. Depending on the amount sprayed on, post-drying in a further stage can be required. The product is then worked up in the aforesaid manner.

In the case of a high heat lability of the spray-dried enzymes, during the inventive methods the control of the product temperature is of particular importance. It should be selected to be as low as possible, since with increasing temperature and/or duration of the spray drying and agglomeration method, the losses in activity increase. Typically, the product temperature in spray drying, that is the temperature of the solid spray-dried powder, is at about 40 to 75° C., in particular lower than about 70° C., frequently lower than 60° C. The longer the residence time in the fluidized bed, the lower the temperature should be selected.

The product temperature during agglomeration and drying in the fluidized bed, that is the temperature of the agglomerate in the fluidized bed, is to be selected low during the relatively long residence time in the apparatus, and is at values of about 30 to 70° C., in particular below 60° C., and preferably below 50° C.

To reduce the residual moisture content further, it can be necessary to carry out a post-drying step. During the post-drying also, the product temperature should be in the abovementioned range, and in particular at 50° C. or below. The post-drying reduces the residual moisture content in the inventive preparations to values of less than about 20% by weight, preferably about 5 to 17% by weight.

Drying during agglomeration and the post-drying is achieved by using preheated feed air. The feed air temperature which can be varied depending on the selected preset product temperature, air rate and spray rate, is generally in a range between 30 and 180° C. The post-drying proceeds at a lower temperature, that is to say in the range from about 35 to 55° C.

The duration of agglomeration is likewise dependent on the size of the batch selected, but is in the range from 30 minutes to a plurality of hours.

f2) Production of the Enzyme Formulation

Using mixing techniques known per se, the spray-dried, if appropriate agglomerated, preproduct (dry enzyme composition) is mixed with the above-described support material. For this the enzyme preparation is added to the support, for example a little at a timer, and this is mixed, if necessary for some time, for example 1 to 5 minutes, until a uniform distribution is achieved. Then the hydrophobic liquid is added. This can be sprayed, added dropwise or poured onto or into the mixture during the mixing operation. After addition is complete, the mixing operation is continued, for example for 5 to 45 minutes, until the oil is uniformly distributed. The resultant product has a very low dust fraction. Further handling steps are usually not required.

Various mixer types are suitable for the mixing, such as, for example, cone and screw mixers (for example from Nauter), plowshare mixers (for example from Lödige), twin-shaft mixers. The mixing times depend on the mixer type selected and can differ.

### g) Food and Feed Compositions

The enzyme formulations produced according to the invention are suitable, in particular, for additizing foods and feeds.

The formulations are particularly suitable as additives to animal feed in a mixture with single-component feeds of plant or animal origin in accordance with the FMV (German feed regulation), such as, for example, secondary cereal products, wheat feed meal, wheat bran; extraction meals, spent grains, melasses-dried beet pulp, fish meal, meat and bone meals; and/or mineral single-component feeds according to FMV, such as for example, carbonates, phosphates, sulfates, propionates. Those which are likewise suitable are cereals, such as wheat, rye, barley, oats, corn, millet or triticale; secondary cereal products (by-products of milling), such as brans, semolina brans, wheat semolina brans, feed meals or middlings; by-products from oil production (extraction meals, expeller meals, cakes); by-products from sugar production (melasses, dried cossettes, feed sugars, pulps, potato starch, corn gluten, wheat gluten); by-products from the fermentation industry, brewers spent grains, yeast, malt germ, brewers spent wash; and also animal and other feeds, such as blood meal, fish meal, pressing juice, potato protein.

### EXPERIMENTAL PART

### Production Example V1

### Xylanase Formulation

a) In an aqueous xylanase concentrate having a dry mass content of about 20 to 35% by weight, a pH in the range of from 3.5 to 5.0 and an activity of 60 000 to 100 000 TXU/g, 10-20% by weight of magnesium sulfate heptahydrate were dissolved, based on the concentrate, at 4-10° C.

b) For spray drying and agglomeration, the enzyme composition produced under a) was sprayed in a laboratory fluidized bed Aeromat type MP-1 from Niro-Aeromatic via a 2-fluid nozzle by the top-spray method. The plastic cone of the fluidized bed had a gas distribution plate diameter of 110 mm and a perforated plate having 12% open surface area. The fluidized bed was charged with an air rate of 50 m³/h and feed air temperatures of 40 to 100° C. The feed air temperature was regulated, so that the product in the fluidized bed maintained a temperature of approximately 45° C. The spraying time was 240 min. The product was subsequently cooled with swirling at 50 m³/h feed air at 30° C.

c) The enzyme composition produced under b) was sieved. Fine material and coarse material were sieved out, so that a usable fraction was obtained having a particle size distribution from 100 μm to 400 μm.

This produced a product having the following characteristic data:

### Composition:

| | |
|---|---|
| Xylanase (dry mass) | 65% by weight |
| Magnesium sulfate (MgSO₄) | 20% by weight |
| Residual moisture | 15% by weight |
| Activity | from 200 000 to 300 000 TXU/g |
| Appearance (microscope) | Agglomerates comprising a |
| | plurality of primary particles |
| Median particle diameter | 171 μm |

d) To produce the enzyme formulation, wheat semolina bran (675.5 g) was charged into a laboratory mixer (Lödige) and homogenized at room temperature and 170 rotations per minute. Under these conditions, 21 g of the enzyme composition produced under c) were added to the mixer and mixed for 5 min. Thereafter, 3.5 g of soybean oil were slowly added dropwise via a pipette and thereafter post-mixed for 30 min.

This produced a product having the following characteristic data:

### Composition:

| | |
|---|---|
| Wheat semolina bran (dry mass) | 90% by weight |
| Enzyme composition (from c)) | 3% by weight |
| Soybean oil | 0.5% by weight |
| Residual moisture | 6.5% by weight |
| Activity | from 5000 to 7000 TXU/g |
| Median particle diameter: | 337 μm |

### Production Example V2

### Glucanase Formulation

a) In an aqueous β-glucanase concentrate having a dry mass content of about 20 to 35% by weight, a pH in the range of from 3.5-5.0 and an activity of 150 000 to 400 000 TGU/g, 10 to 20% by weight of magnesium sulfate heptahydrate, based on the concentrate, were dissolved at 4-10° C.

b) For spray drying and agglomeration, the enzyme composition produced under a) was sprayed in by the top-spray method via a 2-fluid nozzle in a laboratory fluidized bed Aeromat type MP-1 from Niro-Aeromatic. The plastic cone of the fluidized bed had a gas distribution plate diameter of 110 mm and a perforated plate having 12% open surface area. The fluidized bed was impinged with an air rate of 50 m³/h and feed air temperatures of 40 to 100° C. The feed air temperature was controlled in such a way that the product in the fluidized bed maintained a temperature of approximately 45° C. The spray time was 240 min. The product was then cooled to 30° C. with swirling at 50 m³/h feed air.

c) The enzyme composition produced under b) was sieved. Fine material and coarse material were sieved out, so that a usable fraction having a particle size distribution of 100 μm to 400 μm was obtained.

This produced a product having the following characteristic data:

### Composition:

| | |
|---|---|
| Glucanase (dry mass) | 65% by weight |
| Magnesium sulfate (MgSO₄) | 20% by weight |
| Residual moisture | 15% by weight |
| Activity | from 500 000 to 120 000 TGU/g |
| Appearance (microscope) | Agglomerate comprising a |
| | plurality of primary particles |
| Median particle diameter | 167 μm |

d) For production of the enzyme formulation, wheat semolina bran (693 g) was charged into a laboratory mixer (Lödige) and homogenized at room temperature and 170 revolutions per minute. Under these conditions, 3.5 g of the enzyme composition produced under c) were placed in the mixer and mixed for 5 min. Thereafter, 3.5 g of soybean oil were slowly added dropwise via a pipette and thereafter post-mixing is performed for 30 min.

This produced a product having the following characteristic data:

### Composition:

| | |
|---|---|
| Wheat semolina bran (dry mass) | 92.5% by weight |
| Enzyme composition (from c)) | 0.5% by weight |
| Soybean oil | 0.5% by weight |
| Residual moisture | 6.5% by weight |
| Activity | from 1000 to 7000 TGU/g |
| Median particle diameter | 321 μm |

### Production Example V3

### Xylanase/Glucanase Formulation

For production of an enzyme formulation, wheat semolina bran (672 g) was charged into a laboratory mixer (Lödige) and homogenized at room temperature and 170 revolutions per minute. Under these conditions, 21 g of the enzyme composition produced under Production Example V1 c) and 3.5 g of the enzyme composition produced under Production Example V2 c) were added to the mixer and mixed for 5 min. Thereafter, soybean oil was slowly added dropwise via a pipette, and subsequently post-mixed for 30 min.

This produced a product having the following characteristic data:

### Composition:

| | |
|---|---|
| Wheat semolina bran (dry mass) | 89.5% by weight |
| Enzyme composition (from V1 c)) | 3% by weight |
| Enzyme composition (from V2 c)) | 0.5% by weight |
| Soybean oil | 0.5% by weight |
| Residual moisture | 6.5% by weight |
| Xylanase activity | from 5000 to 7000 TXU/g |
| Glucanase activity | from 1000 to 7000 TGU/g |
| Median particle diameter | 328 μm |

### Production Example V4

### Xylanase/Glucanase Formulation

a) An aqueous β-glucanase concentrate having a dry mass content of from about 20 to 35% by weight, a pH in the range of from 3.5-5.0 and an activity of from 150 000 to 400 000 TGU/g was mixed with an aqueous xylanase concentrate having a dry mass content of from about 20 to 35% by weight, a pH in range of from 3.5 to 5.0 and an activity of from 60 000 to 100 000 TXU/g in the ratio 1:8. In the mixture, 10 to 30% by weight of magnesium sulfate heptahydrate, based on the concentrate, were dissolved at 4-10° C.

Subsequently, the enzyme concentrate obtained under a) was further processed as in Production Example V1 in the steps b) to d).

This produced a product having the following characteristic data:

### Composition:

| | |
|---|---|
| Wheat semolina bran (dry mass) | 90% by weight |
| Enzyme composition (from c)) | 3% by weight |
| Soybean oil | 0.5% by weight |
| Residual moisture | 6.5% by weight |
| Xylanase activity | from 5000 to 7000 TXU/g |
| Glucanase activity | from 1000 to 7000 TGU/g |
| Median particle diameter | 343 μm |

### Test Example 1

### Determination of the Dust Value

The dust value (% based on total amount of product) of inventive mixtures is determined with and without addition of oil.

The determination proceeded according to the following method:

Three samples each, each of 10±0.03 g of the solid under test are poured slowly (approximately 2 to 3 seconds) through a falling tube (length=60 cm; diameter=3 cm) into a container (20.2 cm in height, 19.5 cm in width, 19.5 cm in length; a suction tube is situated on a side wall at a height of approximately 13 cm and is mounted at a right angle (90°) to the falling tube). Using an oil pump connected via the suction tube to the container, the resultant dust is sucked out of the container and collected on a filter at a constant rate (15±0.5 l/min) for 1 minute. For this, use is made of a glass vacuum filter (diameter 35 mm, D2, 50 ml) provided with a suitable filter (for example Sartorius glass fiber prefilter, 13 400-37-S; diameter 35 mm). The amount of dust removed by suction is determined using an analytical balance, related to the amount of sample used and expressed as a percentage mean. According to the percentage dust values determined, the dust behavior of the samples is described as follows:

| Dust value [%] | Description |
|---|---|
| 0-0.05 | virtually dust free |
| 0.05-0.25 | slightly dust-forming |
| 0.25-1.00 | dust-forming |
| >1.00 | strongly dust-forming |

### Materials Used:

Xylanase powder (XEA): activity: 229 300 TXU/g; median particle diameter=171; (20% by weight magnesium sulfate heptahydrate); dried in a similar manner to Production Example V1

Wheat semolina bran (WGK) (Hildebrandmühlen); median particle diameter=370 Soybean oil

### Mixing the Samples:

The WGK is charged into the Lödige mixer, the SD powder is added thereto and is premixed at room temperature and 5 min at 170 rpm. The soybean oil is heated to approximately 80° C., slowly added dropwise via a fine pipette and post-mixed for 30 min. In each case 1000 g of mixture are prepared. The dust values determined for various mixtures and also for pure XEA and pure WGK are summarized in the following table:

| | Wheat semolina | SD | Soybean | Theoretical | Dust | | Soybean |
|---|---|---|---|---|---|---|---|
| Sample | bran | powder | oil | activity | value | | oil |
| E5/051 | (g) | (g) | (g) | (TXU/g) | (%) | Notes | (%) |
| Batch 1 | 967.3 | 32.7 | 0.0 | 7500 | 0.081 | slightly dust-forming | 0.0 |
| Batch 2 | 962.3 | 32.7 | 5.0 | 7500 | 0.025 | virtually dust free | 0.5 |
| Batch 3 | 957.3 | 32.7 | 10.0 | 7500 | 0.015 | virtually dust free | 1.0 |
| WGK | pure | — | 0.0 | 0 | 0.120 | slightly dust-forming | 0.0 |
| XEA | — | pure | 0.0 | 229300 | 0.049 | virtually dust free | 0.0 |

A surprisingly significant reduction in dust forming tendency of inventive oil-comprising mixtures is observed.

In addition, after visual examination and also light-microscopy study of the batches studied no differences in separation behavior could be found (results not shown).

### Test Example 2

### Determination of Flowability

The flow behavior of inventive enzyme formulations is determined by known methods. In the prior art, various methods suitable in principle are described (see Schmitt et al., Part. Part. Syst. Charact. 21 (2004) 403-410).

According to the invention, the determination is performed using the Schulze ring shear tester RST.01-pc. The experiment is performed by the method ASTM D6773 (Schulze Ring Shear Tester 2002).

The following test parameters were used:
Storage time of the sample in the measurement cell: 0 h
Temperature: 22° C.
Relative air humidity: 70%
Consolidation force (load): σ₁=11.18 kPa

Using the ASTM D6773 method, a flowability of ff_{c}=8.8 was achieved. Thus the product has high flowability.

1-36. (canceled)

37. A solid enzyme formulation for producing an animal feed, food, or a food supplement comprising a mixture of
a) at least one particulate enzyme composition comprising at least one enzyme and at least one organic or inorganic salt of a monovalent or divalent cation;
b) at least one particulate inorganic or organic support; and
c) at least one hydrophobic liquid;
wherein the ratio of the median particle diameter of said at least one particulate inorganic or organic support to said at least one particulate enzyme composition is in the range of from about 0.125 to about 8 and the mixing ratio of said at least one particulate enzyme composition and said at least one particulate inorganic or organic support is in the range of from about 1:1000 to about 1:5 parts by weight.

38. The solid enzyme formulation of claim 37, comprising
a) a particulate enzyme composition comprising an enzyme in a mixture with at least one organic or inorganic salt of a monovalent or divalent cation; or
b) a particulate enzyme composition comprising at least two enzymes which are different from one another in a mixture with at least one organic or inorganic salt of a monovalent or divalent cation; or
c) at least two different particulate enzyme compositions, wherein each composition comprises at least one different enzyme, wherein said at least one different enzyme in each composition is in a mixture with at least one organic or inorganic salt of a monovalent or divalent cation.

39. The solid enzyme formulation of claim 37, wherein the ratio of the median particle diameter of said at least one particulate inorganic or organic support to said at least one particulate enzyme composition is in the range of from about 025 to about 5.

40. The solid enzyme formulation of claim 39, wherein the median particle sizes of said at least one particulate enzyme composition and said at least one particulate inorganic or organic support are, independent of one another, in the range of from about 50 to about 500 μm.

41. The solid enzyme formulation of claim 37, wherein the mixing ratio of said at least one particulate enzyme composition and said at least one particulate inorganic or organic support is in the range of from about 1:500 to about 1:10 parts by weight.

42. The solid enzyme formulation of claim 37, wherein said hydrophobic liquid is present in an amount in the range of from 0.1 to 5% by weight, based on the total weight of the enzyme formulation.

43. The solid enzyme formulation of claim 37, wherein said at least one organic or inorganic salt of a monovalent or divalent cation is present in the range of from 1 to 30% by weight, based on the total weight of the enzyme composition.

44. The solid enzyme formulation of claim 37, wherein said at least one enzyme is selected from the group consisting of xylanases, glucanases, cellulases, proteases, keratinases, amylases, and mixtures thereof.

45. The solid enzyme formulation of claim 44, wherein said at least one enzyme is selected from the group consisting of endo-1,4-B-xylanases (EC 3.2.1.8), endo-1,4-β-glucanases (EC 3.2.1.4), and mixtures thereof.

46. The solid enzyme formulation of claim 37, wherein said solid enzyme formulation has:
a) a gravimetric dusting value in the range from 0.001 to 0.2%; and/or
b) a bulk density in the range of from 200 to 700 g/l; and/or
c) a flowability ff_{c} (as determined by Schulze ring shear test) in the range of from 3 to 30.

47. The solid enzyme formulation of claim 37, wherein said
a) at least one particulate enzyme composition comprises a mixture of an enzyme selected from the group consisting of xylanases, glucanases, and mixtures thereof with magnesium sulfate, wherein said magnesium sulfate is present in an amount in the range of from about 5 to 25% by weight, based on the total weight of the dry enzyme composition;
b) at least one particulate inorganic or organic support comprises at least one wheat semolina bran support; and
c) at least one hydrophobic liquid is vegetable oil present in an amount in the range of from about 0.1 to 1% by weight, based on the total weight of the enzyme formulation;
wherein the mixing ratio of said at least one particulate enzyme composition to said at least one particulate inorganic or organic support is in the range of from 1:5 to 1:500 and the median particle size of said at least one particulate enzyme composition and said at least one particulate inorganic or organic support is in the range of from about 150 to about 500 μm, said xylanase is present in amount in the range of from about 3000 to about 30,000 TXU/g of formulation, and said glucanase is present in amount in the range of from about 2000 to 20,000 TGU/g of formulation.

48. The solid enzyme formulation of claim 47, wherein said enzyme is a xylanase.

49. The solid enzyme formulation of claim 47, wherein said enzyme is a glucanase.

50. The solid enzyme formulation of claim 47, wherein said enzyme is a mixture of xylanase and glucanase.

51. The solid enzyme formulation of claim 47, comprising two enzyme compositions comprising different enzymes, wherein one enzyme composition comprises a glucanase and the other enzyme composition comprises a xylanase.

52. A method for producing the solid enzyme formulation of claim 37, comprising mixing at least one particulate enzyme composition comprising at least one enzyme and at least one organic or inorganic salt of a monovalent or divalent cation with at least one particulate inorganic or organic support to form a mixture and wetting said mixture with a hydrophobic liquid.

53. The method of claim 52, wherein said at least one particulate enzyme composition comprises
a) a particulate enzyme composition comprising an enzyme in a mixture with at least one organic or inorganic salt of a monovalent or divalent cation; or
b) a particulate enzyme composition comprising at least two enzymes which are different from one another in a mixture with at least one organic or inorganic salt of a monovalent or divalent cation; or
c) at least two different particulate enzyme compositions, wherein each composition comprises at least one different enzyme, wherein said at least one different enzyme in each composition is in a mixture with at least one organic or inorganic salt of a monovalent or divalent cation.

54. The method of claim 52, wherein said at least one particulate enzyme composition is obtained by spray drying or by spray drying and agglomeration of an enzyme-comprising liquid in which at least one organic or inorganic salt of a monovalent or divalent cation is taken up.

55. The method of claim 52, wherein at least two different particulate enzyme compositions, wherein each composition comprises at least one different enzyme, are obtained by spray drying or by spray drying and agglomeration of at least two different enzyme-comprising liquids in which at least one organic or inorganic salt of a monovalent or divalent cation is taken up, and
a) each of the at least two enzyme compositions is mixed with a particulate inorganic or organic support, or
b) a particulate inorganic or organic support is mixed with the at least two enzyme compositions; and
the mixture produced according to a) or b) is wetted with a hydrophobic liquid.

56. The method of claim 54, wherein said enzyme-comprising liquid comprises at least one xylanase, at least one glucanase, or a mixture thereof.

57. The method of claim 52, wherein said at least one organic or inorganic salt of a monovalent or divalent cation is present in the range of from 1 to 30% by weight, based on the total weight of said enzyme composition.

58. The method of claim 52, wherein the ratio of the median particle diameters of said at least one particulate enzyme composition to said at least one particulate inorganic or organic support is in the range of from about 0.125 to about 8.

59. The method of claim 58, wherein the median particle sizes of said at least one particulate enzyme composition and said at least one particulate inorganic or organic support are, independent of one another, in the range of from about 50 to 500 μm.

60. The method of claim 52, wherein the mixing ratio of said at least one particulate enzyme composition to said at least one particulate inorganic or organic support is in the range of from about 1:1000 to 1:5.

61. The method of claim 52, wherein said hydrophobic liquid in an amount in the range of from 0.1 to 5% by weight, based on the total weight of said enzyme formulation.

62. A method for producing a solid enzyme formulation comprising at least one enzyme selected from the group consisting of xylanases, glucanases, and mixtures thereof, comprising
a) spray drying or spray drying and agglomerating at least one enzyme-comprising liquid to give at least one enzyme composition comprising an enzyme selected from the group consisting of xylanases, glucanases, and mixtures thereof, wherein said enzyme is present in said at least one enzyme-comprising liquid in a mixture with magnesium sulfate, wherein said magnesium sulfate fraction is present in a range of from about 5 to about 25% by weight, based on the total weight of the dry enzyme composition;
b) mixing said at least one enzyme composition with a particulate inorganic or organic support to form an enzyme/support mixture; and
c) wetting said enzyme/support mixture with a hydrophobic liquid.

63. The method of claim 62, wherein said at least one enzyme composition comprises
a) at least one xylanase in a mixture with magnesium sulfate; or
b) at least one glucanase in a mixture with magnesium sulfate; or
c) at least one xylanase and at least one glucanase in a mixture with magnesium sulfate; or
d) at least two particulate enzyme compositions different from one another, wherein one composition comprises at least one xylanase and the other composition comprises at least one glucanase, and wherein the enzymes in each composition are in a mixture with magnesium sulfate.

64. The method of claim 62, wherein said at least one enzyme composition is mixed with at least one wheat semolina bran support, wherein the mixing ratio of said at least one enzyme composition to said at least one wheat semolina bran support is in the range of from 1:5 to 1:1000.

65. The method of claim 62, wherein said at least one enzyme composition comprises two different enzyme compositions, which are mixed with at least one wheat semolina bran support, wherein the mixing ratio of said at least one enzyme composition to said at least one wheat semolina bran support is in the range of from 1:5 to 1:1000.

66. The method of claim 62, wherein said hydrophobic liquid is vegetable oil in an amount in the range of from about 0.1 to about 1% by weight, based on the total weight of the solid enzyme formulation.

67. The method of claim 62, wherein the median particle sizes of said at least one enzyme composition and said particulate inorganic or organic support are, independent of one another, in the range of from about 150 to about 500 μm.

68. The method of claim 62, wherein said xylanase is present in an amount in the range of from about 3000 to about 30,000 TXU/g of formulation and/or said glucanase is present in an amount in the range of from about 2000 to about 20,000 TGU/g of formulation.

69. An animal feed, food, or food supplement comprising the solid enzyme formulation of claim 37.

70. An animal feed comprising the solid enzyme formulation of claim 37.

71. The animal feed of claim 71, wherein said solid enzyme formulation is present in an amount in the range of from about 0.001 to about 1% by weight.
